(19) United States
(12) Patent Application Publication
(10) Pub. No.: US 2022/0287302 A1
(43) Pub. Date: Sep. 15, 2022

(54) COMPOSITIONS FOR PATHOGEN INACTIVATION OR PATHOGEN REDUCTION MANAGEMENT

(71) Applicant: Amitava MAZUMDER, Hyderabad (IN)

(72) Inventor: Amitava MAZUMDER, Hyderabad (IN)

(21) Appl. No.: 17/436,884

(22) PCT Filed: May 20, 2021
(86) PCT No.: PCT/IN2021/050491

§ 371 Date: Sep. 7, 2021

(30) Foreign Application Priority Data
Oct. 7, 2020 (IN) 202041043725

(51) Int. Cl. A01N 31/06; A01P 1/00; A01N 25/02

(52) U.S. Cl. CPC A01N 31/06; A01P 1/00; A01N 25/02

(57) ABSTRACT

The present disclosure relates to compositions for pathogen inactivation or pathogen reduction. In particular, compositions for pathogen inactivation or pathogen reduction of air borne, droplet and on surface pathogens. The composition comprises: squaric acid or croconic acid, squaric acid ester or ester of croconic acid, sodium lauryl sulphate, solvent, water, and a base; wherein the base is capable of reacting with squaric acid or croconic acid to form squaric acid dianion or croconic acid dianion. The disclosure also relates to a fumigant composition. The fumigant composition comprises squaric acid ester, solvent, and water. Also, relates to an aerosol solution to increase the effectiveness of conventional non-woven multilayer masks.

### RELATED APPLICATION

This application claims the benefit of Indian provisional application number 202041043725, filed on Oct. 7, 2020 the contents of which are hereby incorporated by reference in their entirety.

### TECHNICAL FIELD

The present disclosure relates in general to compositions for pathogen inactivation or pathogen reduction. Particularly, but not exclusively, the present disclosure relates to compositions for inactivation or reduction or removal of pathogens which are airborne, or pathogens present on the surface.

### BACKGROUND

Pathogens are disease-causing microorganisms. Pathogens are of different kinds such as viruses, bacteria, fungus, and parasites. Pathogens can be found anywhere including in the air, food, and the surfaces that people come in contact with. Pathogen reduction (PR) or pathogen inactivation (PI) means reduction or inactivation or removal of a virus, bacteria, fungus, and/or protozoan pathogen from an object.

Pathogens which are airborne, or pathogens present on the surface and/or within the surface of objects and/or materials cause various symptoms ranging from mild to severe. Severe cases can lead even to death.

The rapidly spreading coronavirus, SARS-CoV-2, and the disease it causes, COVID-19 is still a pandemic across the world. This has greatly damaged human health and economic growth. The most common symptoms of COVID-19 include fever, cough, fatigue, aches and pains, sore throat and difficulty breathing or shortness of breath. The coronavirus is an enveloped virus. Generally, enveloped viruses are less stable in the environment and can be inactivated significantly faster than non-enveloped viruses. To inactivate an enveloped virus an appropriate disinfectant product must be utilized. Research has shown that one of the main transmission pathways of COVID-19 is the airborne and droplets produced by patients which then form biological aerosols. This pathway has also been verified in the spread of other viruses such as SARS viruses and influenza viruses.

Some research show that COVID-19 can survive for more than 3 h in aerosols. Further, some studies show that viral RNA from COVID-19 can be detected in the air. So, controlling the spread of biological aerosols may be the most effective way to inhibit the spread of viruses.

The common measures used currently for protection against airborne viruses include wearing protective mask and clothing, increasing indoor ventilation, and applying disinfectants. However, there are several limitations. For example, N95 respirators, named for their ability to filter 95% or more of tiny 0.3-μm particles to provide protection against airborne pathogens Nonetheless, the 10 Centers for Disease Control and Prevention recommends that health care workers use N95 masks when caring for patients particularly during exposure to procedures that produce high concentrations of aerosols (e.g., intubation, extubation, non-invasive ventilation).

Although surgical masks have lower filtration efficiency than N95 respirators, observational studies have shown no significant benefit of N95 masks over surgical masks for prevention of severe acute respiratory syndrome coronavirus or other respiratory viruses. Importantly, the effectiveness of any mask also depends heavily on its real-world use; variability in mask filtration during clinical care may fluctuate more by mask adherence and fit than by marginal differences in laboratory-based filtration efficiency. In practicality, when worn properly, N95 masks are suffocating, uncomfortable, and difficult to tolerate for long durations. Beyond N95 laboratory-based efficacy, costs have been a major challenge in procurement of adequate mask supply, in addition, most of the existing disinfectants do not have long-term effectiveness.

### SUMMARY

Aspects of the invention include compositions for pathogen inactivation or pathogen reduction. The composition comprises: squaric acid or croconic acid, squaric acid ester or ester of croconic acid, sodium lauryl sulphate, solvent, water, and a base; wherein the base is capable of reacting with squaric acid or croconic acid to form squaric acid dianion or croconic acid dianion.

Also provided, an anti-virus aqueous solution comprising the composition which involves in activation or deactivation of airborne virus thus improving the efficacy of solution for use in any manner of use for example as disinfectant.

In another aspect, provided herein are compositions which can effectively react and neutralize the airborne virus present on surfaces of objects and/or materials like masks thus preventing penetration of such virus through masks.

In yet another aspect, provided herein a multilayer mask comprising the composition. The mask is made of non-woven material with moisture retention capability to retain the aqueous solution for a duration.

In yet another aspect of the present invention, provide herein is an aerosol solution to increase the effectiveness of conventional non-woven multilayer masks.

In yet another aspect, provide herein a fumigant composition. The fumigant composition comprises squaric acid ester, solvent, and water.

In yet another aspect of the present invention, the compositions provided herein have antiviral, antibacterial, antifungal, antimicrobial and/or disinfectant activities.

Other aspects, features and advantages of the invention will become apparent to those skilled in the art from the following description which, taken in conjunction with annexed drawings, discloses exemplary embodiments of the invention.

### BRIEF DESCRIPTION OF THE DRAWINGS

Other objects and advantages of the present invention will become apparent to those skilled in the art upon reading the following detailed description of the preferred embodiments, in conjunction with the accompanying drawings, wherein like reference numerals have been used to designate like elements, and wherein:

Persons skilled in the art will appreciate that elements in the figures are illustrated for simplicity and clarity and may not have been drawn to scale. For example, the dimensions of some of the elements in the figure may be exaggerated relative to other elements to help to improve understanding of various exemplary embodiments of the present disclosure.

FIG. 1 illustrates cryo electronic microscopic (CEM) diagram of SARS-CoV-2.

FIG. 2 illustrates an anti-virus mask 201.

FIG. 3 illustrates cytotoxicity data for the composition according to an embodiment of the present invention.

FIGS. 4-6 illustrate microscopic picture of pseudo-virus infection assay for the composition according to an embodiment of the present invention.

FIG. 7 illustrates cytotoxicity data for the composition according to an embodiment of the present invention.

FIG. 8 illustrates Pseudovirion test data for the composition according to an embodiment of the present invention.

FIG. 9 illustrates virus (SARS-CoV-2) test (qRT-PCR test) for the composition (Formulation-1) according to an embodiment of the present invention.

FIG. 10 illustrates virus (SARS-CoV-2) test (qRT-PCR test) for the composition (Formulation-2) according to an embodiment of the present invention.

FIG. 11 illustrates (TCIDSO) assay report for the composition (Formulation-2) according to an embodiment of the present invention.

FIG. 12 illustrates fogging test report for the composition according to an embodiment of the present invention.

FIGS. 13-15 illustrate NMR of SDBE with MMR attenuated virus without diluent.

FIG. 16 illustrates FTIR data of SDBE.

FIG. 17 illustrates FTIR of MMR protein.

FIG. 18 illustrates FTIR of SDBE+MMR proteins of attenuated virus without diluent

FIG. 19 illustrates powdered XRD (X-ray powder diffraction) data of MMR (RNA virus protein) and MMR protein and SDBE coupling product.

### DETAILED DESCRIPTION

It is to be understood that the present disclosure is not limited in its application to the details of composition set forth in the following description. The present disclosure is capable of other embodiments and of being practiced or of being carried out in various ways. Also, it is to be understood that the phraseology and terminology used herein is for the purpose of description and should not be regarded as limiting.

The use of “including”, “comprising” or “having” and variations thereof herein is meant to encompass the items listed thereafter and equivalents thereof as well as additional items.

Before the present invention is described in greater detail, it is to be understood that this invention is not limited to particular embodiments described, as such may, of course, vary. it is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention, Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention.

For convenience, certain terms used in the specification, examples, and appended claims are collected in this section.

As used herein, the term ‘compound(s)’ comprises the compounds disclosed in the present invention.

As used herein, the term “alkyl” refers to a straight chain or branched saturated hydrocarbon group containing no unsaturation. Where appropriate, the alkyl group may have a specified number of carbon atoms, for example, C₁₋₆ alkyl which includes alkyl groups having 1, 2, 3, 4, 5 or 6 carbon atoms in a linear or branched arrangement. Examples of “alkyl” include, but are not limited to, methyl, ethyl, 1-propyl, 2-propyl, n-butyl, sec-butyl, Cert-butyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl and 4-octyl. The “alkyl” group may be optionally substituted.

As used herein, the term “alkoxy” refers to a straight or branched, saturated aliphatic hydrocarbon radical bonded to an oxygen atom that is attached to a core structure. Alkoxy groups may have one to six carbon atoms. Examples of alkoxy groups include, but are not limited to, methoxy, ethoxy, propoxy, isopropoxy, butoxy, isobutoxy and tert-butoxy.

The term “aryl” refers to monocyclic or fused bicyclic or polycyclic ring system haying the well-known characteristics of aromaticity, wherein at least one ring contains a completely conjugated pi-electron system. Typically, aryl groups contain 6 to 20 carbon atoms (“C₆-C₂₀ aryl”) as ring members, preferably 6 to 14 carbon atoms (“C₆-C₁₄ aryl”) or more preferably, 6 to 12 carbon atoms (“C₆-C₁₂ aryl”). Fused aryl groups may include an aryl ring (e.g., a phenyl ring) fused to another aryl or heteroaryl ring or fused to a saturated or partially unsaturated carbocyclic or heterocyclic ring, provided the point of attachment to the base molecule on such fused ring systems is an atom of the aromatic portion of the ring system. Examples, without limitation, of aryl groups include phenyl, biphenyl, naphthyl, anthracenyl, indanyl, indenyl, phenanthrenyl, and tetrahydronaphthyl.

The term “aryloxy” refers to the group aryl-O. Examples of aryloxy include, but are not limited to, phenoxy, naphthoxy, and the like.

Each embodiment is provided by way of explanation of the invention and not by way of limitation of the invention. In fact, it will be apparent to those skilled in the art that various modifications and variations can be made to the compounds, compositions and methods described herein without departing from the scope or spirit of the invention. For instance, features illustrated or described as part of one embodiment can be applied to another embodiment to yield a still further embodiment. Thus, it is intended that the present invention includes such modifications and variations and their equivalents. Other objects, features, and aspects of the present invention are disclosed in or are obvious from, the following detailed description. It is to be understood by one of ordinary skill in the art that the present discussion is a description of exemplary embodiments only and is not to be construed as limiting the broader aspects of the present invention.

In an embodiment, the present disclosure provides a composition comprising: squaric acid or croconic acid, squaric acid ester or ester of croconic acid, sodium lauryl sulphate, solvent, water, and a base; wherein the base is capable of reacting with squaric acid or croconic acid to form squaric acid dianion or croconic acid dianion. In certain embodiments, the composition has antiviral, antibacterial, antifungal, antimicrobial and/or disinfectant activities. In some instances, the composition has anti-SARS-CoV-2 activity.

The formulations used in Example 1, Example 2 and in fumigating liquid are experimented with minimum and maximum ppm levels of each component in the composition (formulation) for the study of pathogenic inactivation, and varied degrees of efficacy was obtained in every set of test.

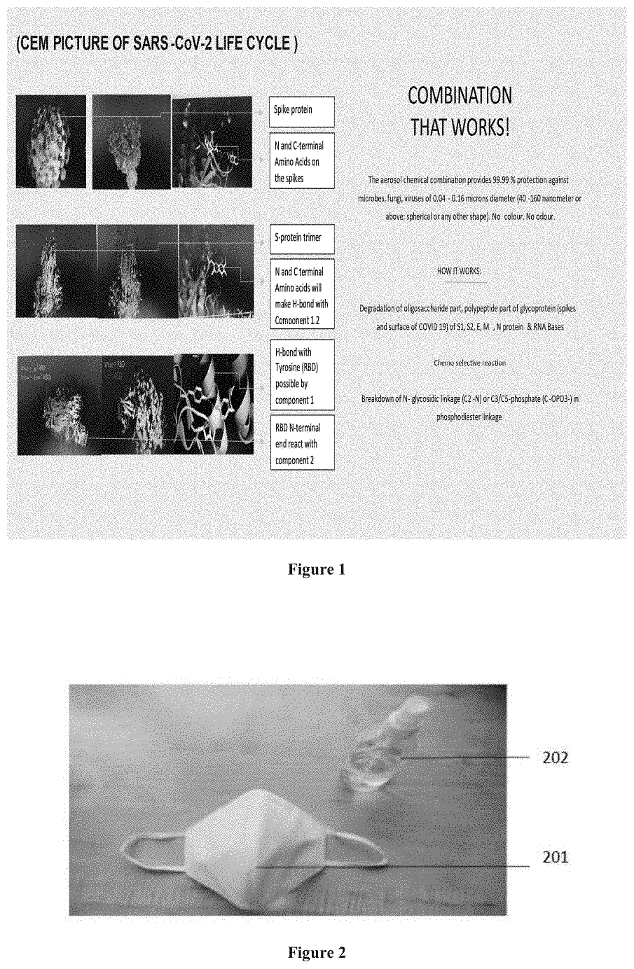

FIG. 1 illustrates cryo electronic microscopic (CEM) diagram of SARS-CoV-2.

### Squaric Acid or Croconic Acid (Component 1)

### Squaric Acid:

Squaric acid, also known as 3,4-dihydroxy-3-cyclobutene-1,2 dione, has the following chemical structure.

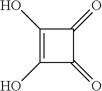

a) Squaric acid has two acidic protons. The high acidity with pKₐ of 1.5 for the first proton and pKₐ of 3.4 for the second proton. It exists in dianion form at pH 7.2 with four point hydrogen bonding sites and highly stable aromatic species.

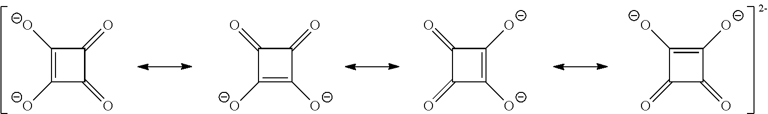

b) Squaric acid forms strong H-bonding with Tyrosine (Y), Serine (S) and other amino acids charged or uncharged amino acid.
c) Tyrosine, Asparagine, Glutamine of receptor binding domain (RBD) of viruses such as SARS-COV-2 make the hydrogen bond with squarate dianion from four possible hydrogen bonding sites strongly, preventing the virus entry in human cell. Otherwise, the amino acids of human ACE 2 namely, tyrosine, glutamine, glutamine (−), Asparagine (+) and Arginine (+) makes hydrogen bonds with the above amino acids of RBD and allows viral entry in host cell. So, use of squarate dianion in the alkaline condition may prevent the entry and the replication of SARS-COV-2 in human body.

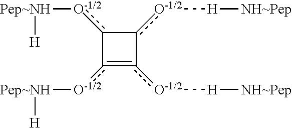

d) With other interior Amino acids which are hydrophilic/hydrophobic amino acids dissolved in the condition of aqueous DMSO, for both RNA and DNA viruses.
e) The hydrolysis of the phosphate esters and removal of the phosphate linkage by Squarate Dianion in presence of mild alkaline medium is also probable reaction which destroys the RNA or DNA structure.

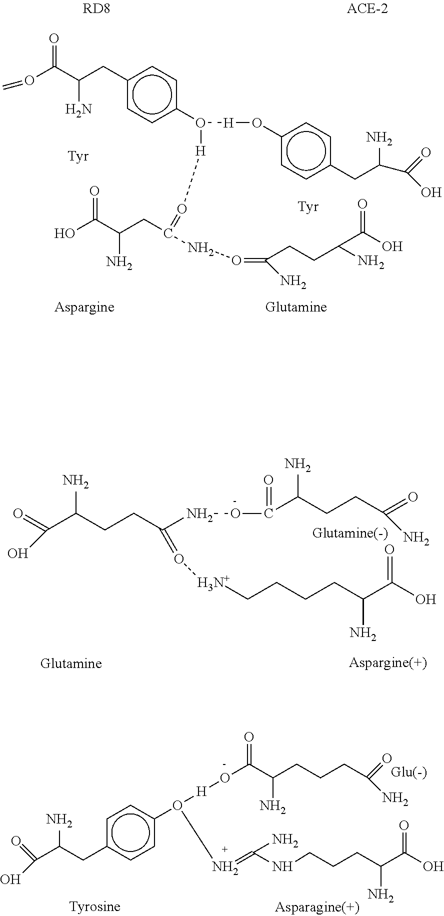

### Croconic Acid:

Croconic acid, also known as 4,5-dihydroxycyclopent-4-enc-1,2,3-triune, has the following chemical structure.

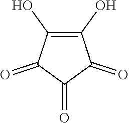

The chemistry of croconic acid is similar to squaric acid in terms of the reactivity, particularly towards nycleophiles.

In certain embodiments, squaric acid or croconic acid is present in the composition in a concentration of from about 0.5 ppm to about 2000 ppm or from about 0.5 ppm to about 1000 ppm or from about 0.5 ppm to about 500 ppm or from about 0.5 ppm to about 250 ppm or from about 0.5 ppm to about 100 ppm or from about 0.5 ppm to about 50 ppm. In some instances, squaric acid or croconic acid is present in a concentration of from about 0.5 ppm to about 2000 ppm or from about 0.5 ppm to about 1000 ppm.

### Squaric Acid Ester or Ester of Croconic Acid (Component 2)

All types of squarate esters like mono/dimethyl squarate, mono/diethyl squarate, mono/diisopropyl squarate, mono/dibutyl squarate or any squaric acid mono/di esters including croconic acid mono/di alkyl or aryl esters and 2 or 2,3 or 2,3,5 or 2,3,5,6 mono/di/tri/tetra alkoxy or aryloxy cyclohexa-2,5-diene-1,4 diene can be used in the present invention.

In certain embodiments, the squaric acid ester is a mono ester or a diester in which two ester groups may he the same or different.

In certain embodiments, the squaric acid ester is squaric acid monoester (also known as squarate monoester). In certain embodiments, the squaric acid monoester ester is an alkyl squarate, an aryl squarate or a combination thereof. Examples of alkyl squarate include, but are not limited to, methyl squarate, propyl squarate, ethyl squarate, butyl squarate, pentyl squarate, hexyl squarate, squarate, octyl squarate, and the like.

In certain embodiments, the squaric acid ester is squaric acid diester (also known as squarate diester). In certain embodiments, squaric acid diester is a dialkyl squarate, a diaryl squarate, an alkyl aryl squarate or any combination thereof. Examples of dialkyl squarate include, but are not limited to, squaric acid dibutyl ester (SADBE), squaric acid diethyl ester (SADEE), squaric acid monohutyl ester (SAMBE), or squaric acid monoethyl ester (SAMEE). In certain embodiments, dialkyl squarate is squaric acid diethyl ester (SADEE). In some instances, dialkyl squarate is squaric acid dibutyl ester (SADBE).

In certain embodiments, squaric acid ester or ester of croconic acid is present in the composition in a concentration of from about 0.5 ppm to about 2000 ppm or from about 0.5 ppm to about 1000 ppm or from about 0.5 ppm to about 500 ppm or from about 0.5 ppm to about 250 ppm or from about 0.5 ppm to about 100 ppm or from about 0.5 ppm to about 50 ppm. In some instances, squaric acid ester or ester of croconic acid is present in a concentration of from about 0.5 ppm to about 1000 ppm or from about 0.5 ppm to about 500 ppm.

### Action of Squaric Acid Ester or Ester of Croconic Acid:

a) SN2 reactions takes place by serine (S) of S-protein and other exterior or interior N-terminal amino acids of DNA and RNA viruses at mild alkaline or close to pH 7.0 from acidic or alkaline range.
b) Specifically, the N-terminal amino acid like Asparagine (Amino acids below pH=7.2), Cysteine (Sulphur atom with negative charge) of RBD can easily attack the squarate esters in SN2 mode, losing its activity and conformation to attack human ACE 2 by the following route in mild alkaline medium.

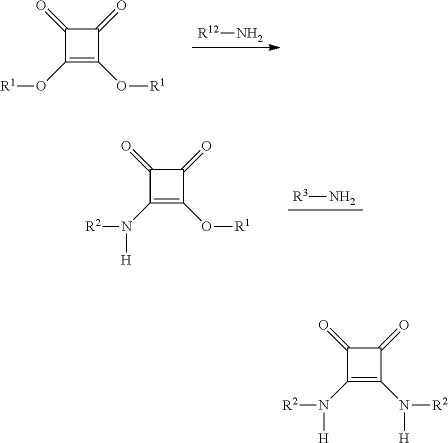

wherein R¹ is glycoprotein/(S-) of Cystine and N of (side chain amino group) asparagine of RBD (for example).

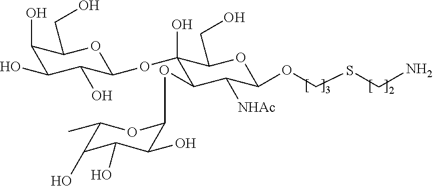

R² is lysine/(S-) of cysteine (Cys145)/N of (side chain amino group) Asparagine (Asn487) or other N-terminal amino acids of glycoprotein of RBD (for example)

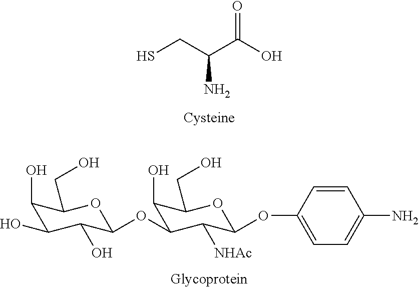 [SMILES: N[C@@H](CS)C(=O)O ; CC(=O)NC1C(Oc2ccc(N)cc2)OC(CO)C(O)C1OC1OC(CO)C(O)C(O)C1O]

Glycoprotein and especially Sugars of glycoproteins make glycoconjugates by squaric acid mediation as shown above and disrupt the backbone of oligosaccharide polypeptide linkage of virus.
c) SN2 reactions done by (hydrophobic) methionine (M) dissolved in aqueous solvent such as aqueous DMSO, of E-protein and other exterior interior amino acids.
d) SN2 reactions done by interior lysine (L) after membrane of E-protein and other exterior interior amino acids.
e) The SN2 reactions by the DNA and RNA bases are highly probable which are linked at C2 of Deoxyribose and Ribose.

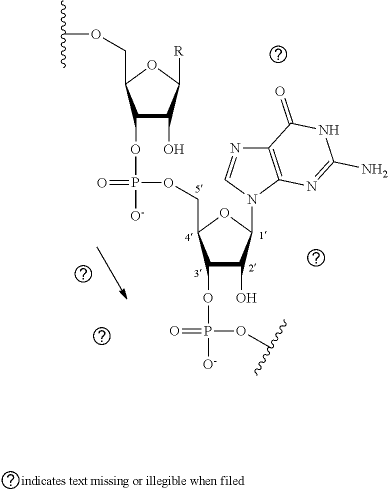

f) Inter nucleotidic squarates as mimic or alternative of phosphate is also quite common occurs by the SN2 reaction mentioned above with diethyl squarate which disrupts the normal constitution of RNA/DNA nucleotide.

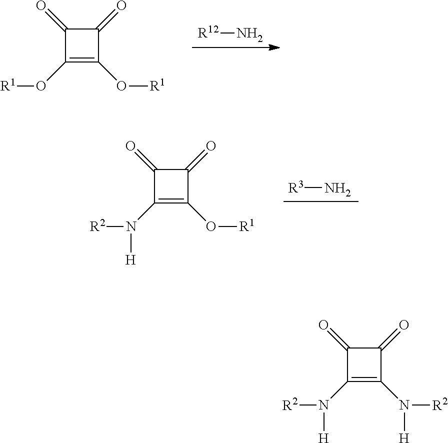

wherein R¹ and R² independently are:

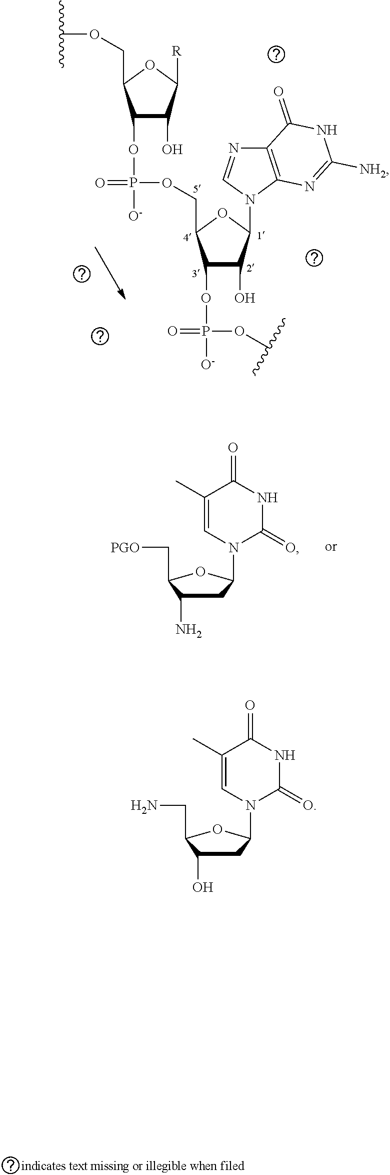

The squarates are as stable as phosphate due to extended conjugation and resultant aromaticity, The N-terminal amino acid end substituted squarate esters resembles phosphate and being stable due to aromatic nature,

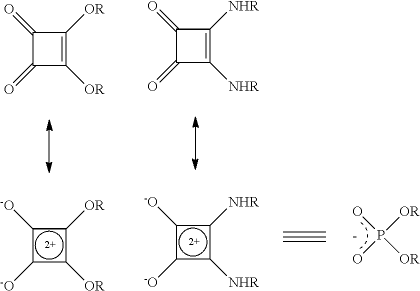

Sodium Lauryl Sulphate (SLS) (Component 3) and its Action:

The composition disclosed herein comprises SLS. In certain embodiments, SLS is present in the composition in a concentration of from about 1 ppm to about 5000 ppm or from about 1 ppm to about 4000 ppm or from about 1 ppm to about 3000 ppm or from about 1 ppm to about 2000 ppm or from about 1 ppm to about 1000 ppm or from about 1 ppm to about 500 ppm or from about 1 ppm to about 250 ppm or from about 1 ppm to about 100 ppm or from about 1 ppm to about 50 ppm. In some instances, SLS is present in a concentration of from about 1 ppm to about 3000 ppm or from about 1 ppm to about 1000 ppm.
a) SLS may cause denaturation and unfolding of the trimer/secondary/tertiary structure of SARS-CoV-2. SLS breaks the intramolecular H-bonding within the single strand/Double strand RNA/Double strand DNA viruses.
b) It has anti-bacterial, microbicidal activities against both enveloped (Herpes simplex viruses, HIV1, Semliki Forest viruses) and nonenveloped viruses (Rota virus, Papilloma viruses, Reovirus and Poliovirus) and antifungal effect.
c) SLS acts as surfactant which lowers the surface tension between ingredients which is why used as cleansing agent and selfcare products and food additives recommended by FDA.
d) It forms charge micelle which by its hydrophobic end surrounds the hydrophobic end of virus and bacteria and other pathogens and brings in aqueous solution.

### Base and Buffer (Component 4):

Base is used to neutralize the squaric acid and to attain the pH of the composition to 7.2-7.5, typically 7.2-7.3. Any base capable of reacting with squaric acid or croconic acid to form squaric acid dianion or croconic acid dianion can be used in the composition. In certain embodiments, base is a carbonate of alkali or alkaline earth metal, bicarbonate of alkali or alkaline earth metal, hydroxide of alkali or alkaline earth metal, or any combination thereof. In certain embodiments, the alkali and alkaline earth metals are selected from the group comprising sodium, potassium, lithium, calcium, barium, and caesium. In some instances, base is sodium hydroxide.

In certain embodiments, buffer is used to maintain the buffering action. In certain embodiments, buffer is a carbonate of alkali or alkaline earth metal, bicarbonate of alkali or alkaline earth metal, hydroxide of alkali or alkaline earth metal, or any combination thereof. In some instances, the buffer is sodicum bicarbonate. In certain embodiments, typically, 1.2 gram per liter (<30 mmoles/lit) of sodium bicarbonate is used to maintain buffering action of the composition. This is non cytotoxic as well as keeps squaric acid in dianionic state keeping options of four-point hydrogen bonding donating sites and replacement of phosphate by squarate mono/dianion in the RNA or DNA nucleotide.

In certain embodiments, base and/or buffer are employed to neutralize the positive ions formed by the N-terminal amino acids of DNA and RNA glycoproteins in S/E/M/N and other non-structural proteins which in consequence reacts with the component used, squaric acid mono/diesters. This helps in breaking down the existing polypeptide linkage of S/E/M/N proteins of DNA and RNA.

In certain embodiments, base and/or buffer in the composition are present in an amount sufficient to maintain a pH of the composition from about 7.2 to about 7.5.

### Solvent (Component 5):

Any suitable solvent can be used in the present invention. For example, any solvent which dissolves squaric acid or squaric acid esters, hydrophilic and hydrophobic ends of lipid bilayer or phospholipid, glycoprotein and protein parts of DNA, RNA and any enveloped viruses into the solution and facilitate the action of the above reagents, and/or non-harmful to human skin and other body organs cn be used in the present invention.

In certain embodiments, solvent is a polar aprotic solvent, polar protic solvent, low polar aprotic solvent, a low polar protic solvent, or a mixture thereof. In certain embodiments, solvent is dimethyl sulfoxide (DMSO), dimethylformamide (DMF), acetone, alcohol, or a combination thereof. Examples of alcohol include, but are not limited to, methanol, ethanol, propanol, isopropanol, butanol, isobutanol, and amyl alcohol.

In certain embodiments. DMSO is used as a solvent. DMSO, for example, readily partition into the bilayer occupying a position beneath the lipid head group reduce bilayer thickness, increase head group area, markedly reduce both the area, compressibility modulus and the bending rigidity of membrane and induce aqueous pore formation which thereby facilitates the entry of the chemicals to interact with virus and deactivate the virus.

Non-enveloped virus can also dissolve in aqueous DMSO to have the effect of the above reagents and neutralize the protein/RNA/DNA.

In certain embodiments, solvent in the composition is present in a concentration of from about 1 ppm to about 5000 ppm or from about 1 ppm to about 4000 ppm or from about 1 ppm to about 3000 ppm or from about I ppm to about 2000 ppm or from about 1 ppm to about 1000 ppm or from about 1 ppm to about 500 ppm or from about 1 ppm to about 250 ppm or from about I ppm to about 100 ppm or from about 1 ppm to about 50 ppm. In some instances, SLS is present in a concentration of from about 1 ppm to about 3000 ppm or from about 1 ppm to about 1000 ppm.

### Water:

Water is added to dissolve the hydrophilic ends of the glycoprotein and protein parts of DNA, RNA and any enveloped or non-enveloped viruses into the solution and facilitate the action of the above reagents and most importantly the hydrolysis of polypeptides and oligosaccharide. In certain embodiments, reagent grade water is used.

In certain embodiments, the composition disclosed herein comprises:
squaric acid or croconic acid in a concentration of from about 0.5 ppm to about 2000 ppm;
squaric acid ester or ester of croconic acid is present in a concentration of from about 0.5 ppm to about 2000 ppm;
p sodium lauryl sulphate a concentration of from about 1 ppm to about 5000 ppm;
solvent in a concentration of from about 1 ppm to about 3000 ppm; and
base in an amount sufficient to maintain a pH of the composition from about 7.2 to about 7.5.

As described above, the formulations used in Example 1, Example 2 and in fumigating liquid are experimented with minimum and maximum ppm levels of each component of the composition (formulation) for the study of pathogenic inactivation, and varied degrees of efficacy was obtained in every set of test.

In certain embodiments, ratio of squaric acid or croconic acid to squaric acid ester or ester of croconic acid in the composition is from about 1:0.5 to about 1:20 or from about 1:0.5 to about 1:15 or from 1:0.5 to about 1:10 or from about 1:0.5 to about 1:5. In some instances, it is from 1:0.5 to about 1:10 or from about 1:0.5 to about 1:5.

The present disclosure also provides a fumigant composition. The fumigant composition comprises squaric acid ester, solvent, and water.

In certain embodiments of the fumigant composition, squaric acid ester is present in a concentration of from about 0.01% by weight to about 2% by weight; and solvent is present in a concentration of from about 0.01% to about 5%.

In certain embodiments of the fumigant composition, squaric acid ester is defined above. In certain embodiments, squaric acid ester is squaric acid monoester or squaric acid diester. In certain embodiments, squaric acid monoester ester is an alkyl squarate, an aryl squarate or a combination thereof; and squaric acid diester is a dialkyl squarate, a diaryl squarate, an alkyl aryl squarate or any combination thereof.

In certain embodiments of the fumigant composition, solvent is as described above. In certain embodiments, solvent is a polar aprotic solvent, polar protic solvent, low polar aprotic solvent, a low polar protic solvent, or a mixture thereof. In certain embodiments, solvent is dimethyl sulfoxide (DMSO), dimethylformamide (DMF), acetone, alcohol, or a combination thereof. Examples of alcohol include, but are not limited to, methanol, ethanol, propanol, isopropanol, butanol, isobutanol, and amyl alcohol. In some instances, solvent is DMSO.

In certain embodiments, compositions disclosed herein are useful for inactivation of airborne pathogens and pathogens on the surface of an object. In certain embodiments, pathogens include viruses, bacteria, fungus, and parasites.

In certain embodiments, the compositions disclosed herein have antiviral activity.

In certain embodiments, the compositions disclosed herein have antibacterial activity.

In certain embodiments, the compositions disclosed herein have antifungal activity.

In certain embodiments, the compositions disclosed herein have antimicrobial activity.

In certain embodiments, the compositions disclosed herein have disinfectant activity. In certain embodiments, the compositions disclosed herein have anti-SARS-CoV-2 activity.

In certain embodiments, the composition disclosed herein are non-cytotoxic.

In certain embodiments, the composition aqueous solution) of the present invention when applied on surfaces like non-woven anti-virus mask neutralizes the activity of DNA/RNA (enveloped/non-enveloped)/fungus/microbes/bacteria.

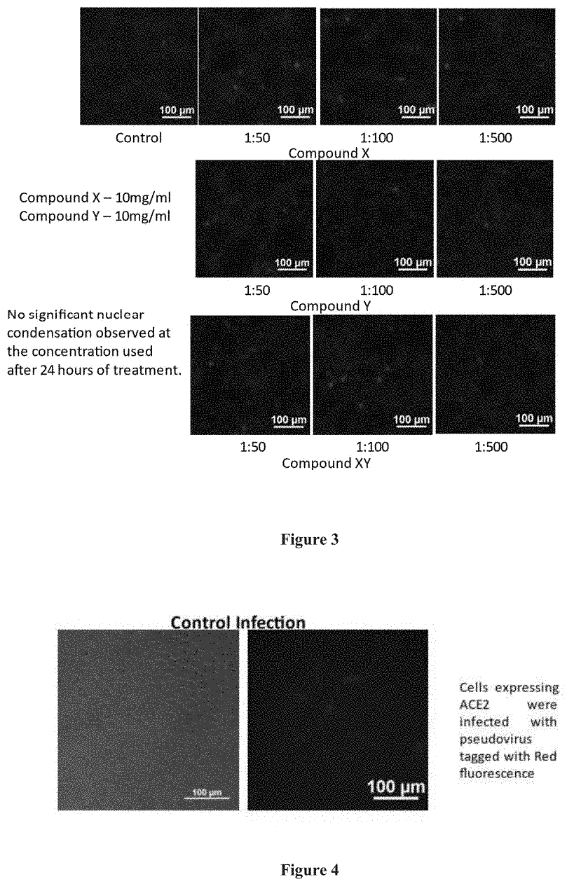

FIG. 2 illustrates an anti-virus mask 201 and an aqueous solution 202 which when applied on the anti-virus mask 201 reacts and neutralizes the activity of virus that is struck in form of particles in the anti-virus mask 201. The anti- virus mask 201 is a four layered mask comprises of Outer layer made of nonwoven cloth, two middle layers consisting of melt blown filters to hold the aqueous solution 202 and the inner layer comprises nonwoven cloth in contact with face. The aqueous solution 202 comprises of: squaric acid at weight concentration of 1.0 gram per 100 ml of aqueous solution, diethyl squarate at weight concentration of 1.0 gram at 100 ml of aqueous solution [Second set experiments done with addition of 1.5 grams squaric acid dibutyl ester keeping other components same in concentration]. DMS0 at weight concentration of 3.0 grams (3.0 ml) per 100 ml of aqueous solution, sodium lauryl sulphate at weight concentration of 0.5 grams per 100 ml of aqueous solution, 0.1M sodium hydroxide was added to near neutralize acid part and 1.2 gram solid sodium bicarbonate was added to make pH 7.2-7.3 as buffer, To make constancy of pH of final aqueous solution and reagent grade water were added to make 100 ml.

The present disclosure also provides a process for preparing the compositions as described above. The process comprises the steps of:
a. dissolving squaric acid or croconic acid in water;
b. dissolving squaric acid ester or ester of croconic acid in a solvent and water;
c. adding sodium lauryl sulphate to a solution of squaric acid in water;
d. adding base to a solution of squaric acid in water to near neutrlistion followed by adding a buffering sodium bicarbonate to maintain a pH of about 7.2-7.3;
e. mixing all the solutions, and making total volume of aqueous solution to 100 ml by adding water; and
f. diluting the whole solution of 100 ml to make 1000 ml of aqueous solution and adjusting pH using base.

In certain embodiments of the process, squaric acid ester, ester of croconic acid, base, solvent, base, buffer, and solvent are same as defined above. In certain embodiments of the process, squaric acid ester is squaric acid monoester or squaric acid diester. In certain embodiments, squaric acid monoester ester is an alkyl squarate, an aryl squarate or a combination thereof; and squaric acid diester is a dialkvl squarate, a diaryl squarate, an alkyl aryl squarate or any combination thereof. In some instances, squaric acid ester is diethyl squarate or dibutyl squarate.

In certain embodiments of the process, solvent is as described above. In certain embodiments, solvent is a polar aprotic solvent, polar protic solvent, low polar aprotic solvent, a low polar protic solvent, or a mixture thereof. In certain embodiments of the process, solvent is dimethyl sulfoxide (DMSO), dimethylformamide (DMF), acetone, alcohol, or a combination thereof. Examples of alcohol include, but are not limited to, methanol, ethanol, propanol, isopropanol, butanol, isobutanol, and amyl alcohol. In some instances, solvent is DMSO. In certain embodiments, water is reagent grade water.

In certain embodiments of the process, base and/or buffer is a carbonate of alkali or alkaline earth metal, bicarbonate of alkali or alkaline earth metal, hydroxide of alkali or alkaline earth metal, or any combination thereof.

In certain embodiments of the process, all the steps are conducted at ambient temperature.

In certain embodiments, typically the composition of the present invention (aqueous solution) which is non-cytotoxic with 7.2 pH is prepared as follows. The process is carried at 25° C. The process comprises the steps of:
a. dissolving 1.0 gram of squaric Acid (Alpha Aesar make) in 20 ml reagent grade water (201);
b. dissolving 1.0 gram of diethyl squarate (Alpha Aesar make) in 3 ml DMSO (MERCK MAKE) and 12 ml reagent grade water (202);
c. adding 0.5 grams of sodium lauryl sulphate (Alpha Aesar make) in solution of squaric acid in reagent water (203);
d. adding 0.1 M solution of sodium hydroxide (Qualigen make) in solution of squaric acid in reagent water to near neutralization and after neutralization of squaric acid pH 7.2-7.3 was attained by adding 0.12% by mass (<30 mmoloes/lit) of sodium bicarbonate to act as buffer (204);
e. mixing all the solution from (201-204) in a beaker and total volume of aqueous solution was made 100 ml by adding reagent grade water (205); and
f. diluting the whole solution of 100 ml to make 1000 ml of aqueous solution (206).

The net chemical reaction which would occur with S-protein and other glycoproteins are given as follows:

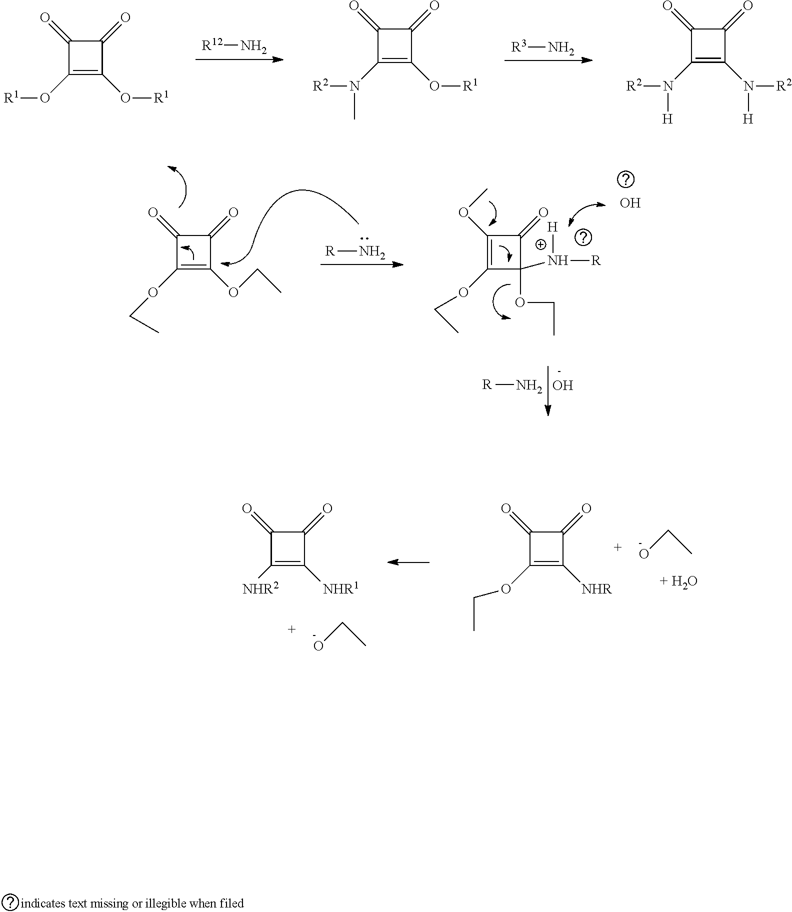

wherein R¹ can be any of the compounds like N-terminal amino acids of Asparagine (Asn 487) or glycoprotein/S(-) of cysteine (Cys 145).

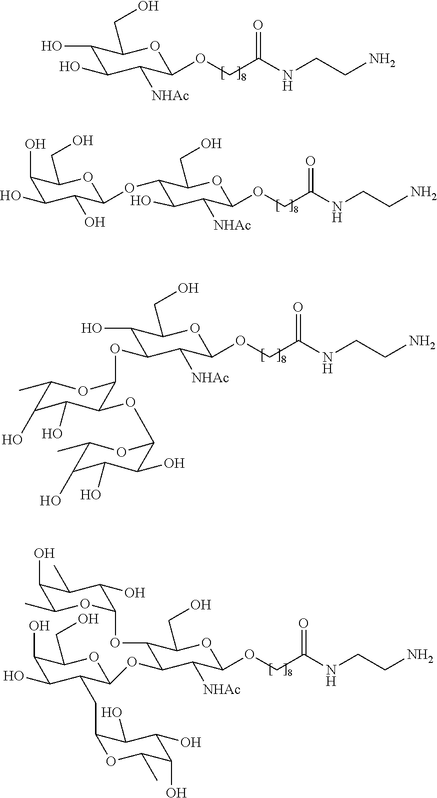

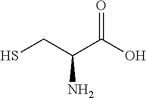

Cysteine can lose proton at pH7.2-7.3
R² can be N-terminal amino acids of Asparagine/S(-) of cystine or glycoprotein of RBD;

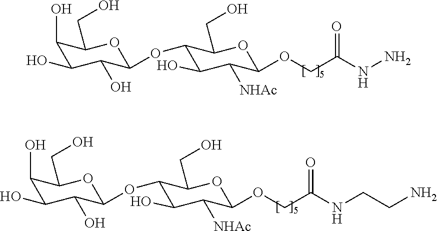

or even amino group connected with nucleotide;

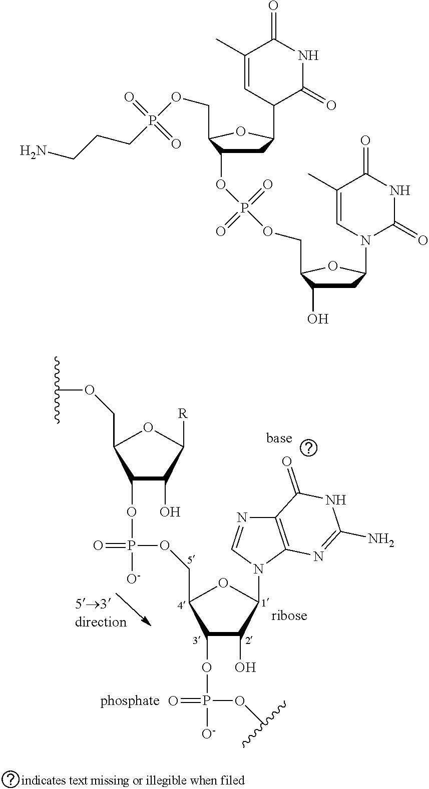

The SN2 reactions occurring with N terminal amino acid like asparagine and S- of cysteine with squarate derivatives are as the following with mono/dialkyl squarates,

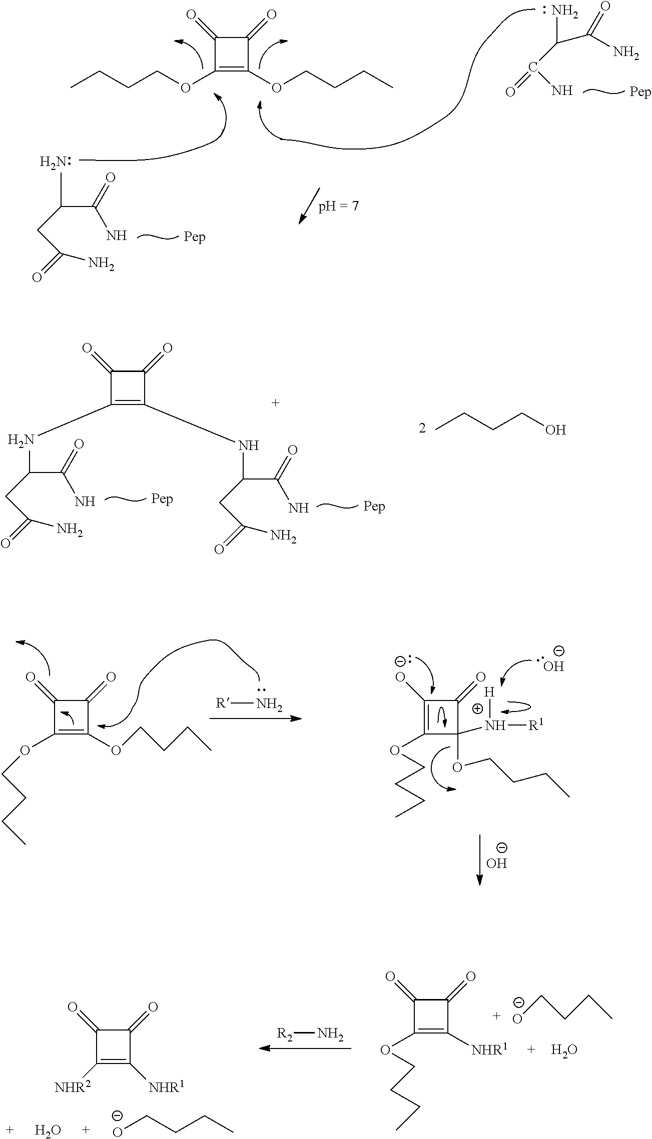

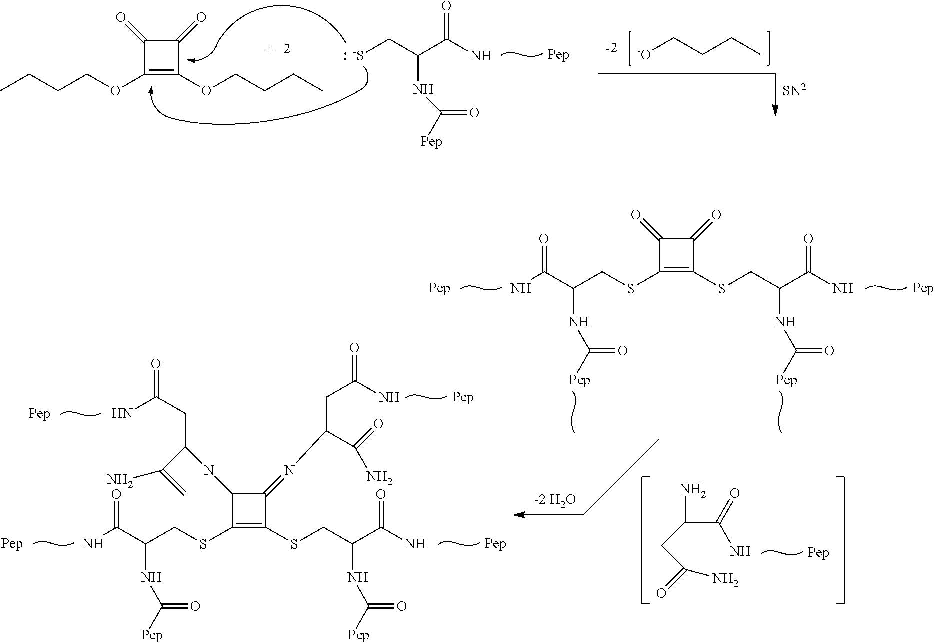

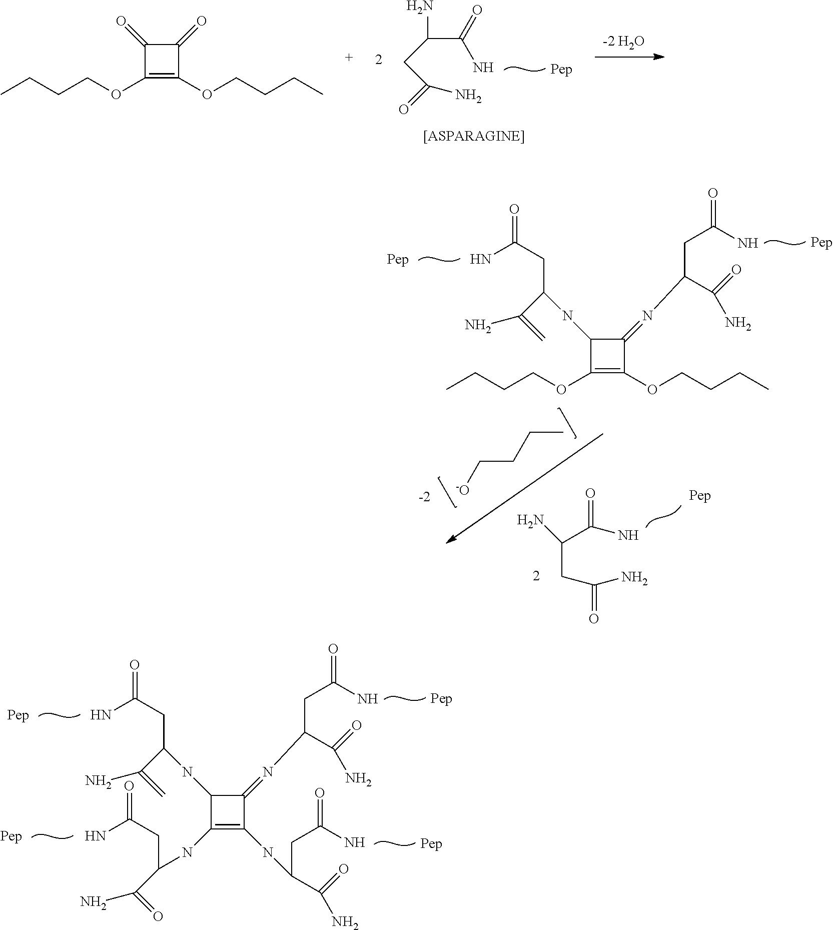

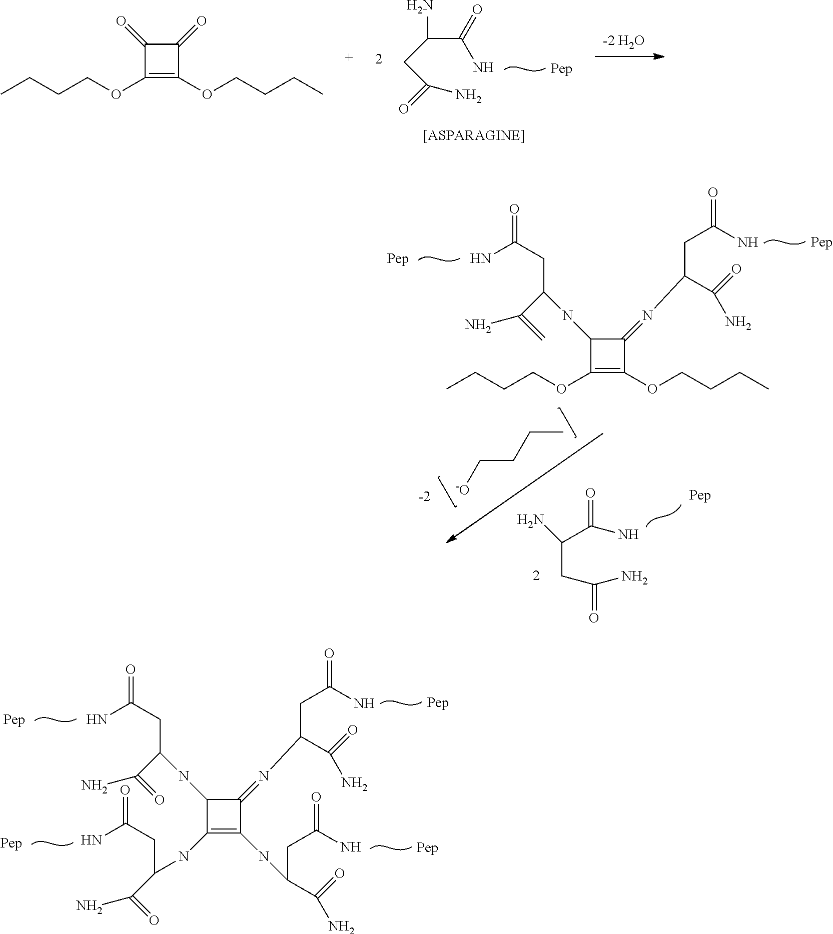

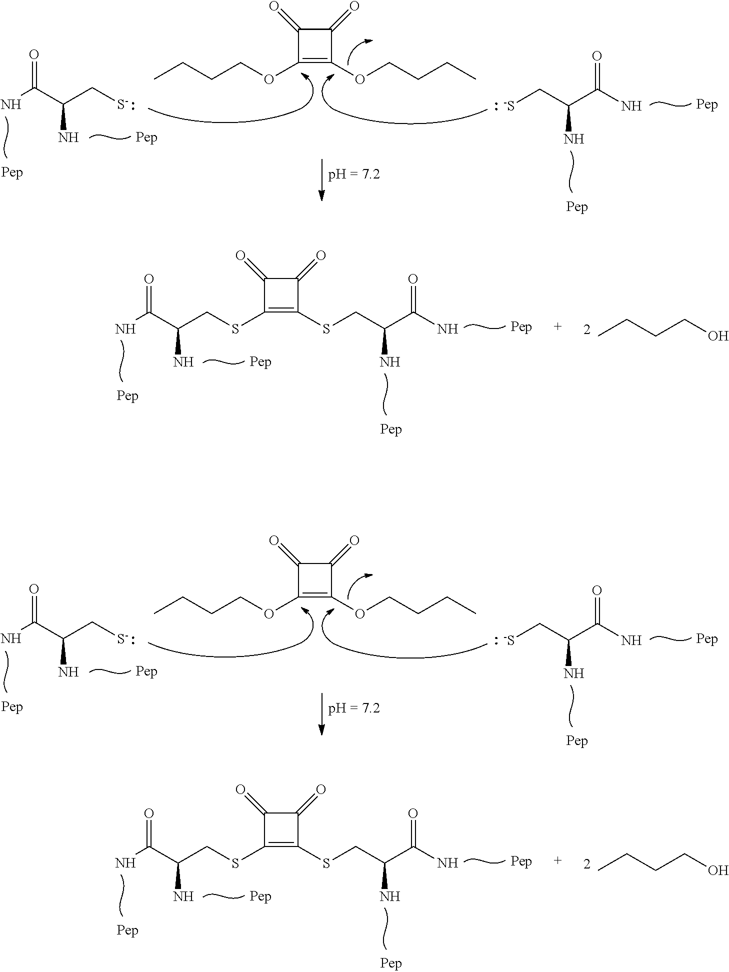

Condensation reaction with 1,2 diketo form may also happen.

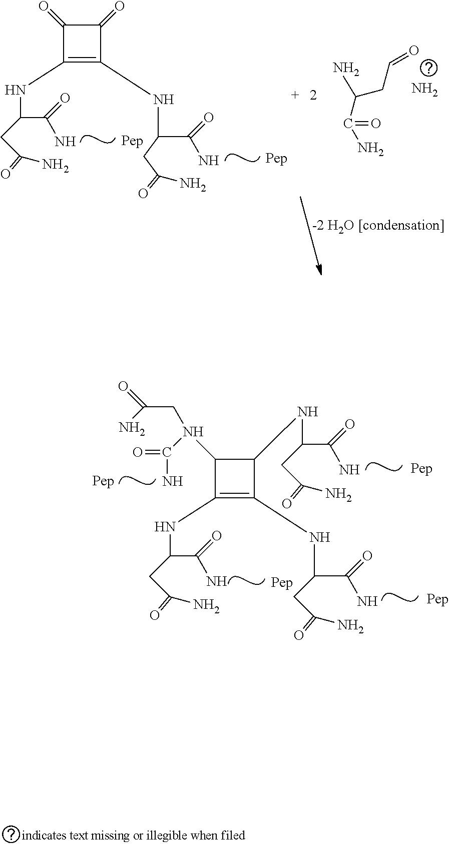

The present disclosure now being generally described, it will be more readily understood by reference to the following examples, which are included merely for purposes of illustration of certain aspects and embodiments of the present disclosure and are not intended to limit the present disclosure in any way.

### EXAMPLES

### Example 1

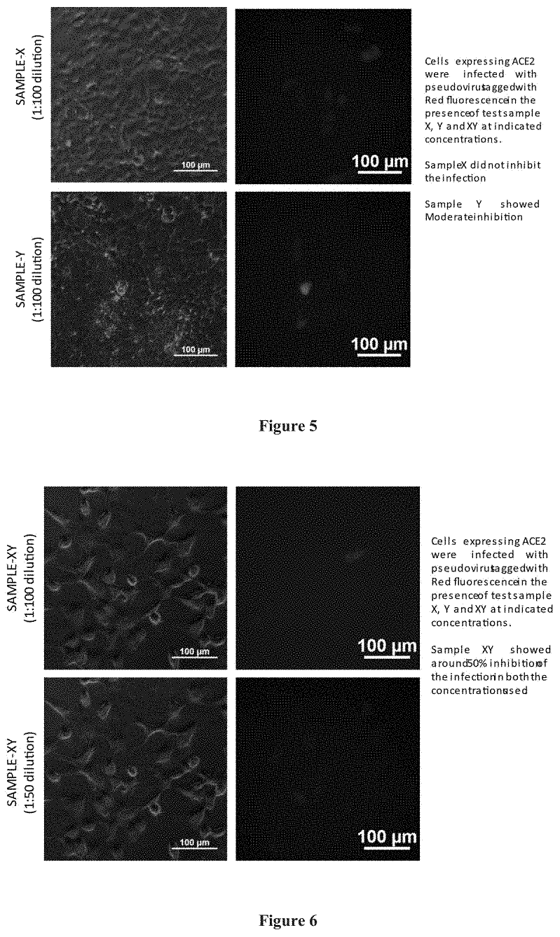

In this example, composition having squaric acid, diethyl squarate, SLS, NaOH NaHCO₃, DMSO and water are prepared. The composition was made using the process depicted in FIG. 3. The composition formulation is as shown below.

Formulation 1: Diethyl squarate 1×10(−1)%, squaric acid 1.0×10(−1)%, DMSO 3×10(−1)%, SLS 0.5×10(−1)%, Required sodium hydroxide to neutralize squaric acid and rest buffering action is done by sodium bicarbonate 0.12% and rest biological grade water.

### Example 2

In this example, composition having squaric acid, dibutyl squarate, SLS, NaOH NaHCO₃, DMSO and water are prepared. This composition formulation was made using similar process conditions as described above with reference to Example 1. The composition formulation is as shown below.

Formulation-2: Dibutyl squarate 1.5×10(−1)%, Squaric acid 1.0×10(−1)%, DMSO 3.0×10(−1)%, SLS 0.5×10(−1)%, Required sodium hydroxide to neutralize squaric acid and rest buffering action is done by Sodium bicarbonate 0.12% and rest biological grade water.

### Example 3: Cytotoxicity Test using Diethyl Squarate and Other Molecules (RGCB, DBT, Govt. of India Kerala)

Part 1:

Assay was Carried Out at Central Cell Line Repository of RGCB, Trivandrum
1) Sample format: Solid/semi solid
2) Compound X (1 gm squaric acid+0.5 gm SLS+NaOH+1.2 gm NaHCO3 pH 7.3)−10 mg/ml
3) Compound Y (1 gram diethyl squarate+3 ml. DMSO)−10 mg/ml
4) Mix of X and Y: Mixed as suggested
5) The dilution used for all 1:50 and 1:100
6) The samples were further diluted in DMEM containing 10% Fetal Bovine Serum to determine the cytotoxicity in DLD 1 cells.

Cytotoxicity Assay using Chromatin Condensation

The DLD 1 cells stably expressing human ACE2 were grown on 96 glass bottom plates and allowed to grow for 24 hours. Then the cells were stained with fluorescent nuclear dye Hoechst 33342 at 5 ug per ml for five minutes. The cells were washed and replaced with fresh medium and maintained in CO₂ incubator at 37° C. for 4 hours. The test samples were incubated with the cells at the indicated concentration. Fluorescent images were captured at 24 h and 48 h using DAPI filter set to visualise cell death using an inverted Fluorescent Microscope Nikon TiE. The images were captured with an EMCCD camera from Andor using NIS element software (Nikon). The cell death was interpreted based on the condensed chromatin compared to the control untreated wells (FIG. 3)

Based on the cytotoxicity none of the sample at dilution at 1:50 or 1:100 induced cell death as shown in the FIG. 3.

### Example 4: Pseudovirion Test (RGCB, DBT, Govt. of India Kerala)

Pseudovirion Assay with diethyl squarate and other mentioned molecules:

The assay is based on the lenti viral backbone expressing Td tomato as a traceable marker. In this assay, stable colon cancer cell DLD 1 expressing human ACE2 was utilised as the SARS permissive cells. The procedure involves transfection of HEK Lenti Cells On vitrogen) with the expression vector encoding Td tomato, a plasmid expressing Spike, and plasmids expressing the minimal set of lentiviral proteins necessary to assemble viral particles (Gag/Pol, Rev). The cells were transfected with the expression vectors prepared via Quiagen Midi prep using lipofectamine 2000 as per the manufacturer's instruction. After 6 h, the cells were replaced with fresh medium containing serum. From the transfected cells, SARS-CoV2-Spike-pseudotyped lentiviral particles were collected at 72 hours and filtered using 0.45 micron filter and used to infect the DLD-hACE2 cells using polybrene as per the standard protocol. The test samples were incubated with speudovirions containing medium at indicated dilutions. The media diluted pseudovirion sample acts as the control. After 48 h the cells were imaged under florescent microscope and cells expressing Td tomato fluorescence were counted and percentage positivity was calculated based on the total number of cells in the field.

| | Average Percentage | |
|---|---|---|
| | of Td tomato | |
| Sample | positive cells | Inference |
| Control No | 22% | S protein pseudotyping generally |
| test sample | | provide 18-22% efficiency with Td |
| | | tomato platform |
| Sample X | 19% | No significant inhibition |
| 1:100 | | |
| Sample Y | 14% | Moderate Inhibition |
| 1:100 | | |
| Sample x + | 11% | Around 50% inhibition |
| y: 1:50 | | |
| Sample x + | 12% | Around 50% inhibition |
| y: 1:100 | | |

Sample X and Y are same as in Example 3.

As per the cytotoxicity results and Pseudovirion assay, the x+y mixture at 1:50 and 1:100 dilution showed 50% inhibition of pseudovirion.

The Microscopic Pictures of Example 3

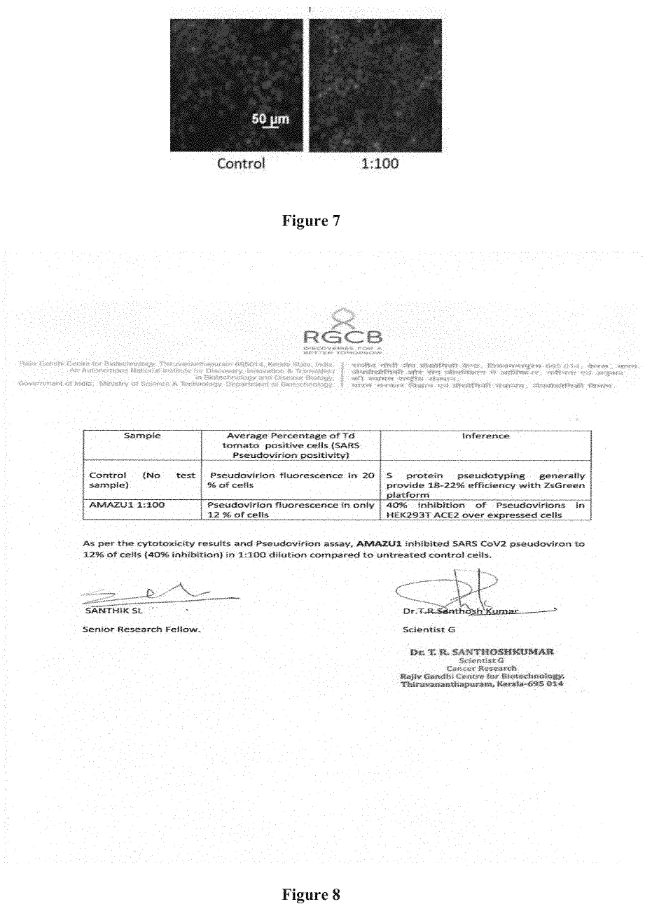

The results are shown in FIG. 3, FIG. 3 shows that no significant nuclear condensation observed at the concentration used after 24 h of treatment. Microscopic picture of pseudo-virus infection assay was shown in FIGS. 4, 5 and 6.

### Example 5

With dibutyl squarate of 2.0 gram, DMSO of 3.0 gram and 995.5 gram DD water the following result was obtained for fumigation purpose.

Part 1:

Assay Carried Out at Central Cell Line Repository of RGCB, Trivandrum

Name of the Test Compound: AMAZU1

The sample “AMAZU1” refers to fumigation liquid composition having the following formulation.

The concentration used: 1:100 dilution of the sample solution (SDBE 1.5 gram, 3 gram DMSO and 995.5 gram water after 1:100 dilution from Initial SDBE and DMSO of 10 ml solution having (1.5 gram SDBE and 3.0 gram DMSO), Thus, percentage by weight was SDBE 0.15%, DMSO 0.3% and rest water at 1:100 dilution.

The samples were further diluted in DMEM containing 10% Fetal Bovine Serum to determine the cytotoxicity in HEK293T cells.

Cytotoxicity Assay using Chromatin Condensation

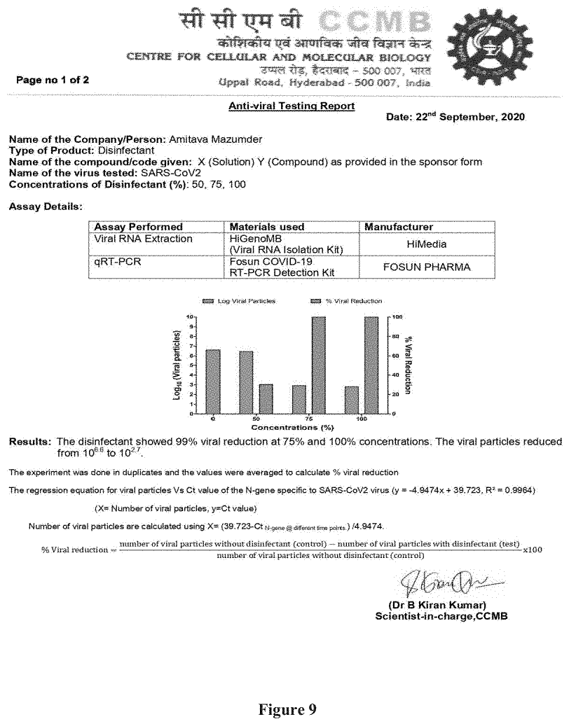

The HEK293T cells stably expressing human ACE2 were grown on 96 glass bottom plates and allowed to grow for 24 hours. Then the cells were stained with fluorescent nuclear dye Hoechst 33342 at 5 ug per ml for five minutes. The cells were washed and replaced with fresh medium and maintained in CO₂ incubator at 37° C. for 4 hours. The test samples were incubated with the cells at the indicated concentration. Fluorescent images were captured at 48 h using DAPI filter set to visualize cell death using an inverted Fluorescent Microscope Nikon TiE. The images were captured with an EMCCD camera from Ardor using NIS element software (Nikon). The cell death was interpreted based on the condensed chromatin compared to the control untreated wells (FIG. 7). Based on the cytotoxicity, the sample at 1:100 dilution showed no cytotoxicity to the HEK293T cells.

### Example 6: Pseudovirion Test (RGCB, DBT, Govt. of India Kerala)

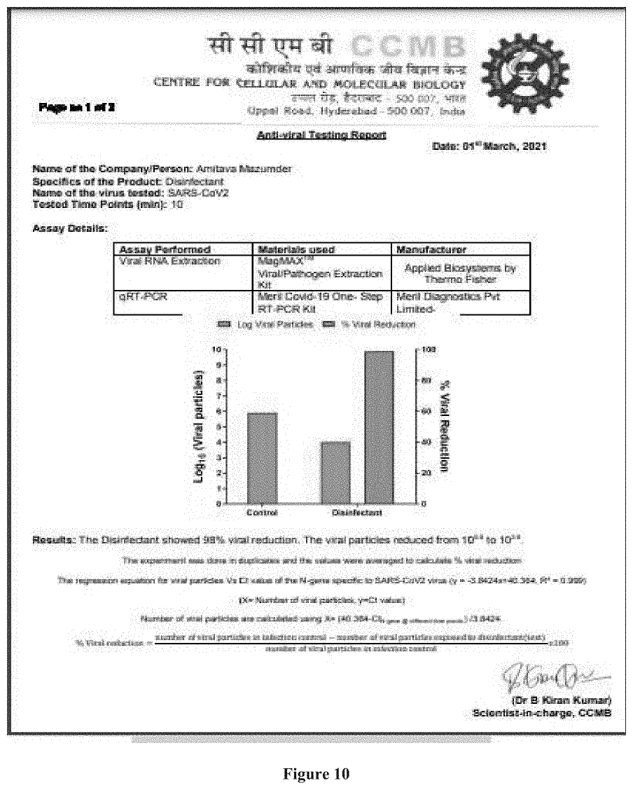

The test was conducted using similar protocol or assay as described above with reference to Example 4 and having SDBE, DMSO and water components as mentioned. The results are shown in FIG. 8.

### Example 7: Live Virus (SARS-CoV-2) Test

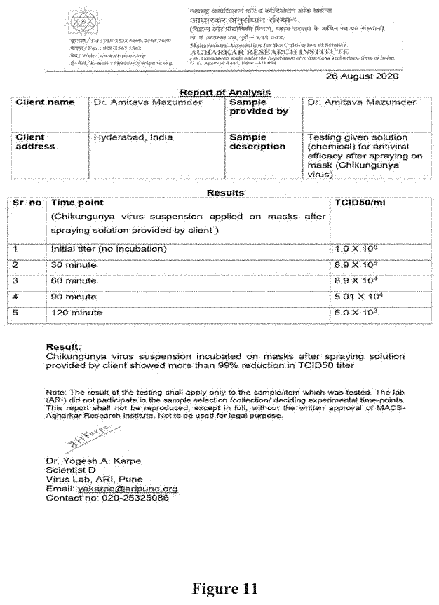

Test was conducted at CCMB, CSIR, Govt. of India with diethyl squarate and other mentioned components as per Formulation-1 in given proportion shows 99.99% efficacy by qRT-PCR test (FIG. 9) in BSL-3 Facility following standard protocol at various concentrations.

### Example 8

Live virus mentioned above at BSL3 facility shows 4 log reduction while the solution was applied on SARS-CoV-2 at 75% and 100% concentration.

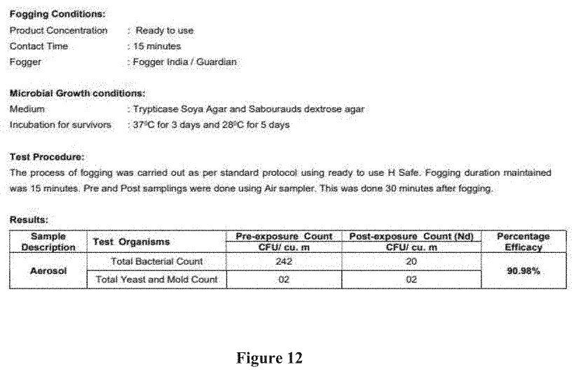

With 100% solution concentration of dibutyl squarate according to Formulation-2, shows 98% viral reduction in 10 minutes (FIG. 10). The standard qRT-PCR technique was used in BSE-3 faxility as per standard International protocol.

### Example 9: Median Tissue Culture Infectious Dose (TCID50) Assay

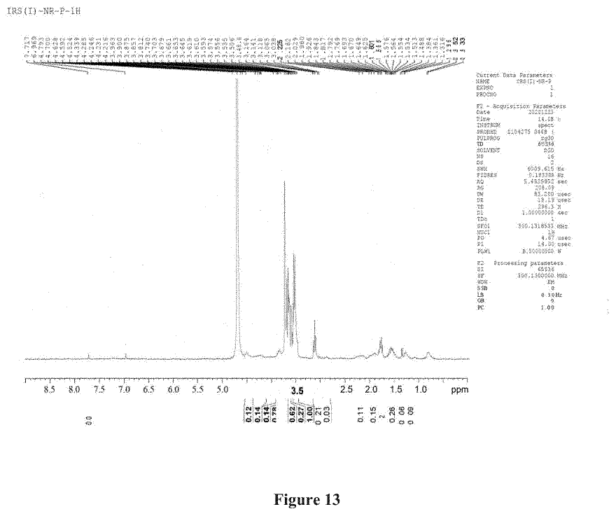

The TCID50 Assay shows >99% efficacy at 120 minutes time point while the solution is sprayed on the mask after viral (Chikanguniya, spherical shaped of diameter 40-60 nm, +SSRNA virus) suspension (FIG. 11). Standard TCID 50 Protocol was used to establish the effect of the SDBE composition of 100% concentration (Formulation-2) on properly configured Mask to find out efficacy at different end points and it shown almost 3 to 5 log reduction.

### Example 10: Effectiveness of Fogging Agent

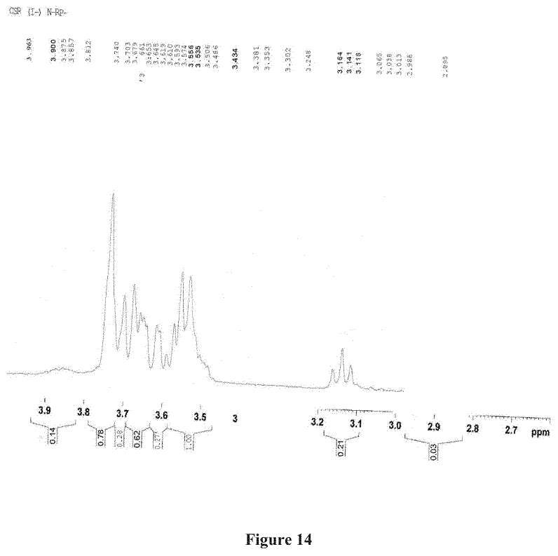

Standard Protocol was used to identify the efficacy of the solution composition in dirty room (indoor). The composition used in this Example is 1.5% dibutyl squarate in 3% aqueous DMSO. The composition resulted in greater than 90% efficacy when used frogging machine as standard (FIG. 12). The result shows that the solution is efficiently clean a dirty room with viruses and bacteria by using fogging machine or fumigator.

### Example 11: NMR Data and Analysis using SDBE with MMR Attenuated Virus without Diluent

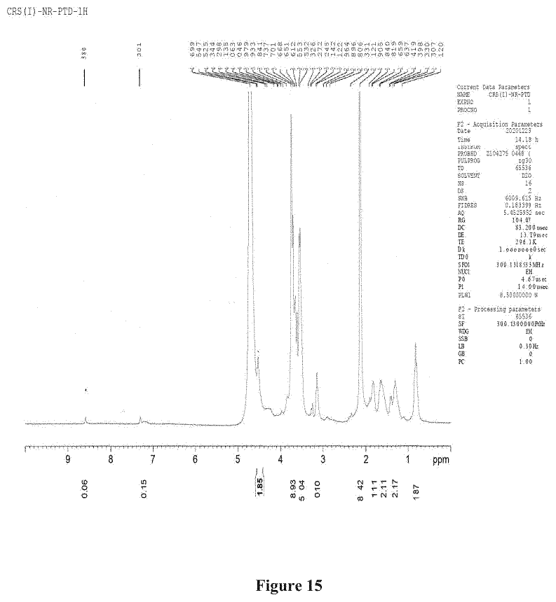

NMR data of SDBE with MMR attenuated virus without diluent is shown in FIGS. 13. 14 and 15.

NMR Analysis:

Down field shift 7.71 to 8.580 and 6.97 to 7.301 clearly shows the NH bonded to —I group —CO— as desired to be established when dibutyl ester (SDBE) reacted with protein (MMR attenuated virus without diluent)) indicates change the configuration and conformation of RNA viruses MMR confirming change in configuration ofvSARS-CoV-2 protein and other protein material.

3.5-4.5 is characteristic alpha C—H bond. Overall data of Hydrogen bonded beta sheet of protein taken (MMR) sovent taken D₂O.

TABLE 1

| In Protein | In butyrate treated protein |
|---|---|
| Not Present | 8.580 |
| Not present | 7.301 |
| 7.71 | Not present |
| 6.97 | Not present |
| Not present | 4.5 |

### Example 12

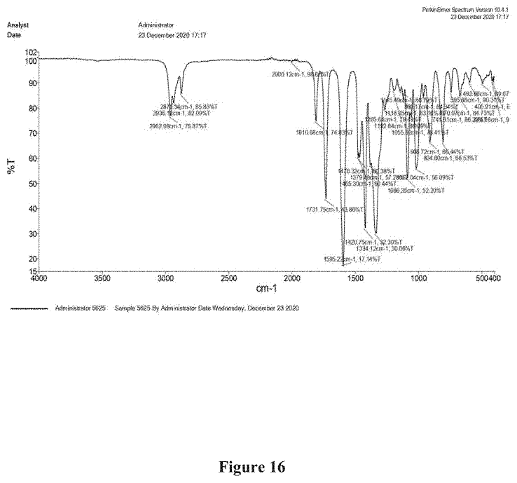

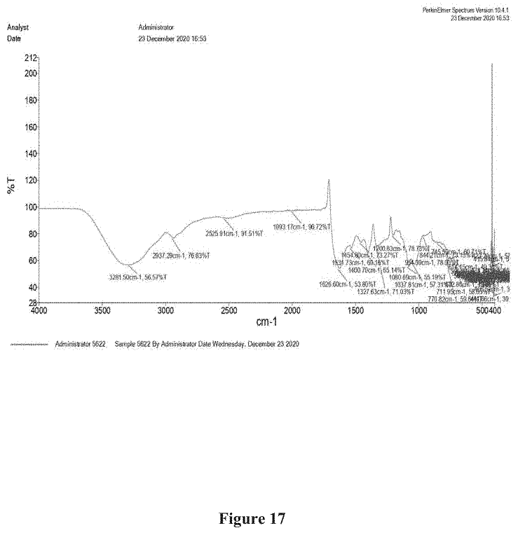

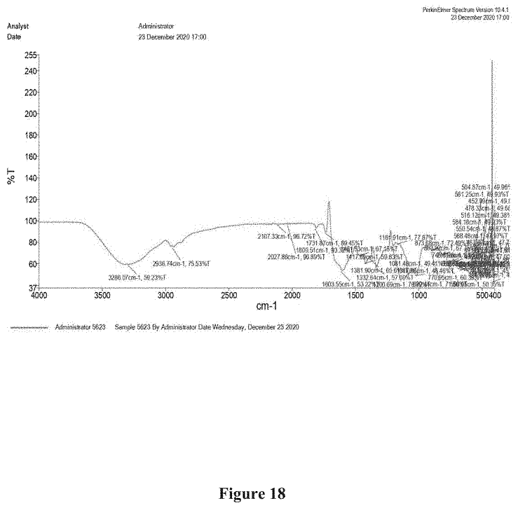

FTIR data of SDBE (FIG. 16) and MMR protein (FIG. 17), and SDBE+MMR proteins of attenuated Virus without diluent (FIG. 18). Reaction of SDBE with protein and RNA can be clearly seen from the FTIR data.

FTER Analysis:

Protein Data (MMR) (FIG. 17)

3281 cm-1 (N—H stretching (Amide A band) which is Hydrogen bonded) 1626.60 cm-1 due to —CONH— (Amide I band) that is NH bonded to —CO that is by —I group proves given protein is a beta sheet 1531 cm-1 N—H bonding

Dibutyl Ester (FIG. 16)

Significant peaks are 1731 (due to —CO—) and Ester group.

Protein Reacted with Dibutyl Ester (FIG. 18)

3268 cm-1 (N—H stretching (Amide-A band), Hydrogen bonded 1731 cm-1, 1631 cm-1 (clear N terminal amino acids of protein bonded to dibutyl ester) as pure dibutyl ester shown peak at 1810cm-1 and 1831 cm-1 due to —CO— part and —COOR part which was present in the original dibutyl ester.

This chemistry is established 1731 cm-1, 1631 cm-1 (Clear N terminal amino acids of protein bonded to dibutyl ester) as it changes the configuration and conformation of any virus having Spike protein/Envelope protein/Membrane protein/Nucleocapsid protein. At pH 10 or above it can act as good conductor as shown in conductivity experiments At pH 7.2 it will act as disinfectant on mask already proven at the blood buffer pH.

### Example 13

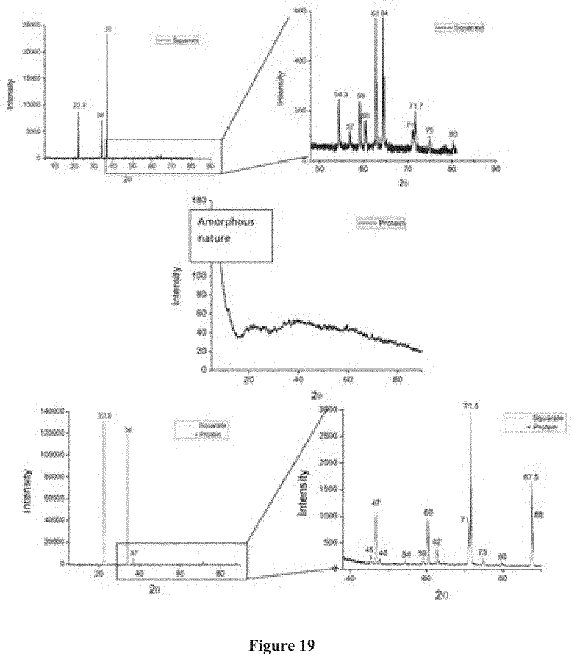

Powdered XRD (X-ray powder diffraction) data of MMR (RNA virus protein) and MMR protein and SDBE coupling product are obtained. The data is shown in FIG. 19. FIG. 19a corresponds to XRD data of MMR (RNA virus protein) and FIG. 19b illustrates MMR protein and SDBE coupling product.

For the proten+sqauarate dianion, a new pak appears at 2θ=45°, 2θ=47° 2θ=48° 2θ=62° 2θ=87° and some old peaks of squarate dianion at 2θ=63°, 2θ=64° 2θ=57° are disappeared. Moreover, the high intensity peak at 2θ=37° which was initially present in squarate dianion is almost disappeared in proten+sqauarate dianion. These differences in the diffraction pattern of proten+sqauarate dianion and squarate indicate that significant structural deformation occurs which are due to hydrogen bonding with sqauarate dianion and protein.

1. A composition comprising: squaric acid or croconic acid, squaric acid ester or ester of croconic acid, sodium lauryl sulphate, solvent, water, and a base; wherein the base is capable of reacting with squaric acid or croconic acid to form squaric acid dianion or croconic acid dianion.

2. The composition as claimed in claim 1, wherein the composition has a pH of about 7.2-7.5.

3. The composition as claimed in claim 1, wherein squaric acid or croconic acid is present in a concentration of from about 0.5 ppm to about 2000 ppm.

4. The composition as claimed in claim 1, wherein squadic acid ester or ester of croconic acid is present in a concentration of from about 0.5 ppm to about 2000 ppm.

5. The composition as claimed in claim 1, wherein sodium lauryl sulphate is present in a concentration of from about 1 ppm to about 5000 ppm.

6. The composition as claimed in claim 1, wherein solvent is present in a concentration of from about 1 ppm to about 3000 ppm.

7. The composition as claimed in claim 1, wherein base is present in an amount sufficient to maintain a pH of the composition from about 7.2 to about 7.5.

8, The composition as claimed in claim 1, wherein a ratio of squaric acid or croconic acid to squaric acid ester or ester of croconic acid is from about 1:0.5 to about 1:20.

9. The composition as claimed in claim 1, wherein squaric acid ester is squaric acid monoester or squaric acid diester.

10. The composition as claimed in claim 1, wherein squaric acid monoester ester is an alkyl squarate, an aryl squarate or a combination thereof; and squaric acid diester is a dialkyl squarate, a diaryl squarate, an alkyl aryl squarate or any combination thereof.

11. The composition as claimed in claim 1, wherein base is a carbonate of alkali or alkaline earth metal, bicarbonate of alkali or alkaline earth metal, hydroxide of alkali or alkaline earth metal, or any combination thereof.

12. The composition as claimed in claim 1, wherein solvent is a polar aprotic solvent, polar protic solvent, low polar aprotic solvent, a low polar protic solvent, or a mixture thereof.

13. The composition as claimed in claim 1, wherein solvent is dimethyl sulfoxide (DMSO), dimethylformamide (DMF), acetone, alcohol, or a combination thereof.

14. A fumigant composition comprising squaric acid ester, solvent and water.

15. The fumigant composition as claimed in claim 14, wherein squaric acid ester is present in a concentration of from about 0.01% by weight to about 2% by weight; and solvent is present in a concentration of from about 0.01% to about 5%.

16. The fumigant composition as claimed in claim 14, wherein squaric acid ester is squaric acid monoester or squaric acid diester.

17. The fumigant composition as claimed in claim 16, wherein squaric acid. monoester ester is an alkyl squarate, an aryl squarate or a combination thereof; and squaric acid diester is a dialkyl squarate, a diaryl squarate, an alkyl aryl squarate or any combination thereof.

18. The fumigant composition as claimed in claim 14, wherein solvent is dimethyl sulfoxide (DMSO), dimethylformamide (DMF), acetone, alcohol, low polar protic solvent or low polar aprotic solvent or any combination thereof.

19. The composition as claimed in claim 1, wherein the composition has antiviral, antibacterial, antifungal, antimicrobial and/or disinfectant activities.

20. The composition as claimed in claim 19, wherein the composition has anti-SARS-CoV-2 activity.
